(19) United States
(12) Patent Application Publication
(10) Pub. No.: US 2020/0188426 A1
(43) Pub. Date: Jun. 18, 2020

(54) WOUND MANAGEMENT METHOD

(71) Applicant: Prevent-Plus, LLC, St. Louis, MO (US)

(72) Inventors: Julie Marie Morris, Wildwood, MO (US); Jeffrey Steven Elbl, Festus, MO (US)

(21) Appl. No.: 16/713,922

(22) Filed: Dec. 13, 2019

Related U.S. Application Data
(60) Provisional application No. 62/779,821, filed on Dec. 14, 2018.

(51) Int. Cl. A61K 31/765; A61M 35/00; A61K 33/20; A61K 9/70; A61K 33/00

(52) U.S. Cl. CPC A61K 31/765; A61M 35/30; A61K 33/20; A61B 5/445; A61K 33/00; A61M 2202/0225; A61K 9/7007

(57) ABSTRACT

A method for wound management in the prevention of bioburden and/or biofilm includes conducting an initial risk assessment protocol with regard to a wound, washing the wound and surrounding skin, applying a polyethylene carbonate composition on and around the wound, eradicating any organisms as methylene chloride of the polyethylene carbonate composition evaporates and polyethylene carbonate polymer of the polyethylene carbonate composition polymerizes, and allowing time for formation of a clear elastomeric film barrier over the wound and around the wound.

### CROSS REFERENCE TO RELATED APPLICATION

This application claims the benefit of U.S. Patent Application Ser. No. 62/779,821, entitled “WOUND MANAGEMENT METHOD,” filed Dec. 14, 2018, the content of which is incorporated herein in its entirety.

### BACKGROUND OF THE INVENTION

### 1. Field of the Invention

The present invention relates to a wound management method. In particular, the present invention relates to a method for the elimination of bioburden in and around a wound in order to prevent biofilm. The method results in blocking oxygen entry to help stimulate hypoxia driven wound healing and lowering the pH by releasing carbon dioxide beneath an occlusive dressing to promote healing. The method also results in creation of an elastomeric dry film covering to protect wounds against water, dirt, and microorganisms, thus eliminating the presence and preventing the migration of potential biofilm producing organisms into the wound bed.

### 2. Description of the Related Art

Biofilms, or organized colonies of bacteria (aerobic and anaerobic), fungi, or yeast, commonly form heterogeneous clusters of organisms on living and fomite materials by secreting extracellular polymeric substances (EPS). These substances protect the microorganisms from factors such as immunologic defenses, nutrient limitations, and antiseptic or antibiotic agents (Ramasamy M & Lee J. Review Article: Recent Nanotechnology Approaches for Prevention and Treatment of Biofilm-Associated Infections on Medical Devices. BioMed Research International. Vol. 2016, Article ID 1851242, 17 pages (1-17) http://dx.doi.org/10.1155/2016/1851242). It has been reported that 99% of all microbes living in the biosphere are capable of forming biofilms (Bhunu B, et al. Inhibition of biofilm formation in Mycobacterium smegmatis by Parinari curatelifolia leaf extracts. BMC Complementary and Alternative Medicine. 2017; 17(285)1-10.). Biofilm can form on any wound when planktonic bacteria are not quickly killed by the patient's own immune system, exogenous antiseptics, or antibiotics. Biofilms can delay wound healing and increase the risk of infection for patients. Because the biofilm protects the microorganisms from the body's natural immune response, it can be difficult for patients to heal on their own. As the body tries to fight the biofilm through an inflammatory response, the body may actually help the biofilm by providing nutrition in the form of exudate (Wolcott R D, Rhoads D D. A study of biofilm-based wound management in subjects with critical limb ischaemia. J of Wound Care. 20008;117(4):145-155). This creates a situation in which the body is ineffectively fighting biofilms while damaging healing tissue and delaying wound healing. Repairing the epidermis is a highly controlled and efficient system involving four broad steps: hemostasis, inflammation, proliferation/migration (re-epithelialization), and tissue remodeling (Guo S, Dipietro L A. Factors Affecting Wound Healing. J Dent Res 2010; 89(3):219-229). Disruption of any of these steps can result in a non-healing wound. Despite only being ^{˜}0.1 mm thick, the epidermis is a complex system acting as the first line of defense for the body. It is critical for the host to repair this barrier, i.e. re-epithelialization, as quickly as possible to restore homeostasis and protection. The presence of microorganisms can significantly delay wound closure.

While antibiotics have been used in preventing biofilm, they are effective only against metabolically active bacteria, which explains why they are not effective against biofilm-associated bacteria, many of which are metabolically inactive. Since antibiotics only kill metabolically active microbes by inhibiting metabolic enzyme systems, inactive bacteria not using these systems may not be sensitive to antibiotics.

Topical antimicrobials have also been used, but some topical antimicrobial agents can have deleterious effects on healthy cells in or adjacent to the wound (cytotoxicity), which may contribute to poor wound healing. Silver, for example, has been shown to be non-specific in its mode of action, killing both bacteria and host keratinocytes and fibroblasts, leading to delayed healing. In addition, not all topical agents have a broad antimicrobial spectrum, which may limit their clinical utility, and bacterial resistance to certain antimicrobial agents has been documented.

### SUMMARY OF THE INVENTION

It is, therefore, an object of the present invention to provide a method for wound management in the prevention of bioburden and/or biofilm. The method comprises conducting an initial risk assessment protocol with regard to a wound, washing the wound and surrounding skin, applying a polyethylene carbonate composition on and around the wound, eradicating any organisms as methylene chloride of the polyethylene carbonate composition evaporates and polyethylene carbonate polymer of the polyethylene carbonate composition polymerizes, and allowing time for formation of a clear elastomeric film barrier over the wound and around the wound.

It is also an object of the present invention to provide a method for wound management including the step of releasing CO₂ into the wound as the polyethylene carbonate polymer of the polyethylene carbonate composition polymerizes and forms the elastomeric film barrier.

It is another object of the present invention to provide a method for wound management including the step of providing a barrier to the flow of O₂ into and from the wound after the elastomeric film barrier is formed.

It is a further object of the present invention to provide a method for wound management including the step of visually inspecting the wound, on a daily basis, through the elastomeric film barrier to identify potential indicators of bioburden, biofilm and/or maceration.

It is also an object of the present invention to provide a method for wound management including the step of inspecting the elastomeric film barrier for leakage and loosening of edges.

It is another object of the present invention to provide a method for wound management wherein the step of conducting an initial risk assessment includes visually inspecting to identify existing bioburden and prior development of biofilm, determining non-healing status of the wound, identifying personal characteristics leading to susceptibility to the development of bioburden and/or biofilm, and identifying potential medical procedures conflicting with conventional wound treatment protocols.

It is a further object of the present invention to provide a method for wound management wherein the step of washing further includes cleaning and drying the wound and surrounding skin.

It is also an object of the present invention to provide a method for wound management wherein the step of applying includes covering an area that extends at least one and one-quarter inches beyond edges of the wound with the polyethylene carbonate composition.

It is another object of the present invention to provide a method for wound management wherein the polyethylene carbonate composition includes polyethylene carbonate polymer dissolved in methylene chloride.

Other objects and advantages of the present invention will become apparent from the following detailed description when viewed in conjunction with the accompanying drawings, which set forth certain embodiments of the invention.

### DESCRIPTION OF THE PREFERRED EMBODIMENTS The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

The present invention provides a wound management system in the form of a method for eliminating bioburden and preventing biofilm in wounds through the application of a microbicidal liquid polymer matrix composed of polyalkylene carbonate polymer, in particular, polyethylene carbonate polymer dissolved in methylene chloride. The present method provides a non-antibiotic protective measure method to eliminate microbes from the wound and surrounding area. In fact, the present method offers a mechanism for eliminating harmful organisms, including resistant organisms, such as, MRSA, Pseudomonas, and Candida. The present method also offers a mechanism for jump starting and enhancing the healing of wounds, providing a transparent elastomeric barrier for wounds, and offers an alternative wound barrier that may be used alone or in conjunction with a variety of dressings. The elastomeric barrier (being non-metallic) also offers the advantage of compatibility with various imaging devices such as MRI, CT scans, or X-Rays.

As those skilled in the art will certainly appreciate, bioburden is understood to refer to the microorganism (e.g. bacteria, virus, fungi) load on a surface, for example, a wound; in particular, the concentration of microorganisms on the surface prior to sterilization. Biofilm is a structured community of microorganisms where cells stick to each other, as well as wound surfaces. The microorganisms are commonly genetically diverse. Biofilms are commonly highly tolerant to antibiotics and biocides. As such, and as contemplated in accordance with the present invention, eradication of bioburden has a positive effect in preventing the formation of biofilm and ultimately in the enhancement of wound healing.

As will be explained below in greater detail, a topically applied composition of polyethylene carbonate polymer and methylene chloride in a fluid form (“polyethylene carbonate composition”) is employed as a mechanism in the prevention of biofilm. As will be appreciated based upon the following disclosure, the polyethylene carbonate composition eliminates bioburden on clean dry, non-infected wounds by killing any microbes the composition comes into contact with through the lysing of cell membranes. As the methylene chloride evaporates and the polyethylene carbonate forms the elastomeric film barrier that protects the clean dry, non-infected wound from harmful external elements, carbon dioxide infused into the liquid polymer composition during the manufacturing process is released. This carbon dioxide effectively lowers the pH below the area of application, that is, the beneath the elastomeric film barrier, creating an environment more closely resembling that of normal human skin. Normal skin pH generally ranges between a pH of 4-6 (acidic), while the pH of the skin is disturbed in wounds where the underlying tissue comes in contact with normal body fluids having a pH around 7.4. This lowering of the pH under the area of application can effectively reduce microbial load on the wound surface (Schneider L A, et al. Influence of pH on wound-healing: a new perspective for wound-therapy? Arch Dermatol Res. 2007; 298:413-420). Lowering of the pH in experimental models has shown to improve fibroblast migration and DNA synthesis (Lengheden A & Jansson L. pH effects on experimental wound healing of human fibroblasts in vitro. Eur J Oral Sci. 1995; 103:148-155).

Another benefit of the elastomeric film barrier is a jumpstarting of the healing process. As an occlusive dressing, impermeable to oxygen, the elastomeric film barrier helps to create an initial hypoxic environment. In the initial phase of wound healing, platelet derived growth factor (PDGF) relies on radical oxygen species (ROS) activity. ROS activity is initiated by wound hypoxia. Hypoxia is also critical in the beginning phase of wound healing for induction of many cytokines such as TGF-β, VEGF, TNF-α, and endothelin-1 (Rodriguez P G, et al. The Role of Oxygen in Wound Healing: A Review of the Literature. Dermatol Surg. 2008; 34:1159-1169). As capillary function is restored in the wound bed (that is, the base portion of the wound site), oxygen delivered by the capillary system is contained by the occlusive dressing. The oxygen is, therefore, available to aid in collagen formation, angiogenesis, and fuel the overall healing process (Bishop A. Role of oxygen in wound healing. Journal of Wound Care. 2008; 17(9):399-402).

As briefly discussed above, the polyethylene carbonate composition of the present method is composed of a polyalkylene carbonate polymer, in particular, polyethylene carbonate polymer, dissolved in methylene chloride. In accordance with a preferred embodiment, the polyethylene carbonate polymer is QPAC® 25 as manufactured by Empower Materials. QPAC® 25 is a poly (ethylene carbonate) solid polymer, and is an amorphous, clear, readily processed plastic with long term mechanical stability. Polyethylene carbonate polymers such as QPAC® 25 also provide the ability for the binding of nanoparticles within a polymer matrix. QPAC° 25 has a molecular weight from approximately 50,000 to 200,000 and exhibits the following characteristics:

| Property | Value |
|---|---|
| Density (g/cm³) | 1.42 |
| Refractive Index | 1.470 |
| Decomposition Temperature (° C.) | 220 (estimate) |
| Glass Transition Temperature (° C.) | 0-10 |
| Heat of combustion (cal/gm) | 3,753 |
| Heat of formation (cal/mol) | −88,726 |
| Molar Mass of repeating unit | 88.1 |

In accordance with a preferred embodiment, the polyethylene carbonate composition is DuraDerm®, a 510K FDA cleared medical device indicated for providing a covering over minor wounds and scrapes that are clean and dry. DuraDerm® consists solely of organic polyethylene carbonate polymer (7.5%-10% by weight) dissolved in methylene chloride organic solvent (90%-92.5% by weight). The formulation eradicates any organisms (bacteria, fungi, viruses) it comes in contact with through lysing of the cell membranes thereof. This is a result of the methylene chloride's activity against an infinite number of organisms. In particular, testing has shown that methylene chloride functions to kill microbes (bacteria, fungi, and viruses) on contact, that is, the methylene chloride functions as a microbicidal agent. As will be appreciated based upon the following disclosure, methylene chloride functions in the manner described above while it is in liquid form and prior to evaporation thereof as the polymer film is formed. The general composition of DuraDerm® is disclosed in U.S. Pat. No. 6,909,027, entitled METHOD OF FORMING AN IN-SITU FILM DRESSING AND THE COMPOSITION OF THE FILM-FORMING MATERIAL and U.S. Pat. No. 7,119,246, entitled METHOD OF TREATING ACNE, both of which are incorporated herein by reference.

After killing microbes on and around the clean dry, non-infected wound area, the methylene chloride of the polyethylene carbonate composition evaporates leaving an elastomeric film barrier that blocks the entry of external oxygen and harmful elements from getting into the clean dry, non-infected wound and thereby protects the clean dry, non-infected wound from harmful elements, such as water, dirt, and germs. By preventing the entry of external oxygen into the clean dry, non-infected wound, the film barrier jump starts the healing process.

The polyethylene carbonate elastomeric film barrier formed after application of the polyethylene carbonate composition is clear, elastomeric, and protects in difficult regions where flexing, bending and creasing skin occurs. Further still, the polyethylene carbonate film is biodegradable and non-metallic. The clear polyethylene carbonate film barrier forms in less than one minute.

Still further, the polyethylene carbonate polymer of the polyethylene carbonate composition is infused with CO₂ as a result of the process of making polyethylene carbonate polymer. In particular, and as those skilled in the art will appreciate, polyethylene carbonate polymer is a copolymer of carbon dioxide and ethylene oxide. When polyethylene carbonate polymer is combusted in an oxygen or air medium, water and carbon dioxide emerge as combustion products.

While the water evaporates with the methylene chloride as the polyethylene carbonate composition dries and the polyethylene carbonate elastomeric film barrier forms, the carbon dioxide released by the polyethylene carbonate polymer is released into the clean dry, non-infected wound. This lowers the pH within the clean dry, non-infected wound and creates a healing environment. As the capillary system is restored within the wound bed, oxygen, through capillary action, becomes readily available to the clean dry, non-infected wound creating an environment encouraging vascular and endothelial growth. The polyethylene carbonate film barrier traps the oxygen and retains it under the polyethylene carbonate film barrier. This oxygen serves as the fuel for re-epithelialization, or wound healing.

In addition, to the readily appreciated consequences of enhancing the healing process, the provision of an environment conducive to wound healing also furthers the goals of killing bioburden and preventing biofilm. In particular, it has been shown that the microbial content within a wound decreases as the wound heals.

Application is commonly accomplished using a cotton tip applicator. The polyethylene carbonate composition is applied on and around the clean dry, non-infected wound area. Momentary stinging may occur upon initial application. The polyethylene carbonate elastomeric film barrier commonly remains intact for one to three days or longer depending on exposure to rubbing, flexing, or soap and water. This polyethylene carbonate film barrier is resistant to degradation by water alone, but can be easily removed with the combination of soap and water or it can be gently peeled off starting at the outer edges. The film barrier adheres well to intact skin, while resisting adherence to newly formed epithelium, and thus can easily be peeled away, or gently washed away with soap and water without disrupting new tissue growth.

While a preferred polyethylene carbonate composition is disclosed above in accordance with a preferred embodiment, it is appreciated polyethylene carbonate polymers can be produced with properties that range from soft elastomeric with low glass transition temperatures (15° C. to 25° C. to 40° C.), to hard stiff polymers with high glass transition temperatures(Tg), e.g. 132° C. Intermediate properties can be produced by chemical (terpolymers) and physical (blends) means. As such, the characteristics of the polyethylene carbonate composition can be varied to suit specific needs within the spirit of the present invention.

Further still, and considering the unique characteristics of polyethylene carbonate polymer used in accordance with the present invention to allow for nanoparticle binding, it is contemplated the polyethylene carbonate composition of the present invention may be used to form microbicidal liquid polymer matrix in which medically effective nanoparticles are bound. Polyalkylene carbonates, in particle, polyethylene carbonates, allowing for nanoparticle binding allow for a faster and more targeted delivery of the matrix to the wound, killing both free-floating and biofilm-associated microbes (bioburden) that may be sitting on the wound. The same can be said for the quicker mechanism of protecting and promoting the wound healing. These polyalkylene carbonates allowing for nanoparticle binding also have the ability to get in fine creases of the skin due to the viscosity of the polyethylene carbonate composition to allow for a more thorough distribution of the polyethylene carbonate composition. As such, the present method is not limited to the use of methylene chloride, as the methylene chloride could also be used in future applications in combination with other biocides, bioengineered living cell therapies, or dyes. Still further, it is contemplated various compounds useful in assisting wound healing could be integrated with the composition, for example, collagen, various growth factors, arginine, antibiotics, antiseptics, etc. The microbicidal liquid polymer matrix discussed above is also compatible with electrospinning. This is all due to nano-binding characteristic of the family of polyethylene carbonates used in accordance with present invention, for example, QPAC® 25.

The physical and chemical properties of this family of nano-binding polyethylene carbonate polymers which are utilized in accordance with the present invention are:
broad spectrum activity against all major microorganisms;
not known to be allergic, unlike Silver and Iodine;
nontoxic to host cells, unlike Silver and Iodine;
not known to be contraindicated for use with enzymatic debridement agents, unlike Silver and Iodine;
maintain a moist wound environment;
film is produced as water-based emulsions which releases microbicidal agent rapidly in a sustained manner;
transparent film placed over wounds allows for wound monitoring without dressing removal;
prevents desiccation and allows cell proliferation to continue;
low cost;
quick drying with no odor;
elastomeric film, which is softened by the skin and body temperature causing flow of the polyethylene carbonate and promoting conformability to body shapes in motion;
barrier to outside water, dirt, and bacteria;
elastomeric barrier can last for days;
film lowers pH and promotes healing;
soluble in a wide range of solvents from low boiling to high boiling;
low glass transition temperature of polyethylene carbonates, e.g. 20° C. to 25° C.;
polyethylene carbonate film is biodegradable and non-metallic;
polyethylene carbonate film is non-metallic which offers the advantage of compatibility with various imaging devices such as MRI, CT scans, or X-Rays unlike silver; and
binding nanoparticles and can be used for electrospinning, which allows for fast delivery of medicants.

In accordance with one embodiment, the polyethylene carbonate composition is as follows: a base solution of polyethylene carbonate (preferably OPAL® 25) is dissolved in methylene chloride to provide a solution in which the polyethylene carbonate is present in a concentration of 7.5%-10% by weight based upon the solution. The polyethylene carbonate composition should be stored in a glass or Teflon lined container, as it is known that methylene chloride reacts with plastics.

The use of polyethylene carbonate as the polyalkylene carbonate polymer in accordance with the present invention results in a polyethylene carbonate composition with excellent oxygen barrier properties, low glass transition temperature (Tg) of about 22° C., very high elongation and recovery, flexibility and elasticity, which provides excellent conformity and protection to irregular body shapes. The low Tg, permits body skin temperatures to soften the polymer further and better conform to irregular shapes, increasing the patient's comfort and providing excellent protection to the treatment area. Film thickness can be from about 0.25 mils to greater than about 3.0 mils, e.g., about 3.5 mils.

In certain cases, external oxygen may be desired and, therefore, polypropylene carbonate can be used, since it is not a good oxygen barrier. For example, and in accordance with another embodiment, a base solution of polypropylene carbonate having a glass transition temperature (Tg) of 40° C. is dissolved in methylene chloride to provide a solution in which the polyethylene carbonate is present in a concentration of 7.5%-10% by weight based upon the solution.

By blending polyethylene carbonate polymers, e.g., polypropylene carbonate and polyethylene carbonate, either physically or chemically (terpolymer), intermediate properties can be obtained to optimize treatment.

There are no other polymer families that can incorporate the unique broad range combination of physical/chemical properties obtainable with this recently developed family of polymers, polyalkylene carbonates. They can be “tailored” to fit the application, thereby providing a better healing system, reducing scarring which adds to patient comfort, and reducing costs.

While polyethylene carbonate is disclosed in accordance with a preferred embodiment, a variety of alkylene substituents can be employed in the polyalkylene carbonate polymer of the present invention to alter the properties of the final polymer or polymers; however, it is preferred to utilize polymers having lower alkylene substituents containing less than about 10 carbon atoms. Typically, polyalkylene carbonate carbonates having from about 2 up to about 9 carbon atoms are employed. Most frequently, ethylene, propylene, or butene are used as the alkylene substituent in the polyalkylene carbonate polymers of the present invention.

In accordance with the present invention the method for preventing bioburden is achieved in the following manner:
1. Conducting initial risk assessment protocol, including, but not limited to, visual inspection to identify existing bioburden and prior development of biofilm, determining non-healing status of wound, identifying personal characteristics leading to susceptibility to the development of bioburden and/or biofilm, identifying potential medical procedures conflicting with conventional wound treatment protocols (for example, imaging procedures such as MRI, CT scans, or X-Rays), etc.
2. Washing the wound and surrounding skin with soap and water, and thereafter cleaning and drying the wound and surrounding skin.
3. Ensuring the area is free of creams, lotions, or ointments, before applying the polyethylene carbonate composition.
4. Applying the polyethylene carbonate composition on and around the wound (preferably with a cotton tip applicator), covering an area that extends at least one and one-quarter inches beyond the edges of the wound. Momentary stinging may occur.
5. Eradicating any organisms (bacteria, fungi, viruses) as the methylene chloride evaporates and the polyethylene carbonate polymer polymerizes.
6. Allowing time for the formation of the clear elastomeric film barrier (film forms in less than one minute).
7. Release of CO₂ into the wound while the elastomeric film barrier is forming.
8. Providing a barrier to the flow of O₂ into and from the wound after the elastomeric film barrier is formed.
9. Inspecting elastomeric film barrier to ensure the formation of a complete, continuous, flexible covering of the wound and surrounding area that is not susceptible to cracks, tears, or otherwise being damaged through movement of the body in the area of the wound.
10. Visually inspecting the wound, on a daily basis, through the elastomeric film barrier to identify potential indicators of bioburden, biofilm and/or maceration, including, but not limited to, undesirable coloration in and around the wound, discharge coming from the wounds, etc.
11. Inspecting the dry elastomeric film barrier covering frequently for leakage and loosening of the edges.
12. Although the elastomeric film barrier itself remains intact under normal use, the method may include the step of applying additional layers if the dry elastomeric film barrier begins to slough off or peel around the edges in one to three days depending on external exposure such as soap and water, rubbing, etc., thereby obviating the need to remove the original elastomeric film barrier and expose the wound to potentially harmful microbes in the environment.
13. If it is, however, desired to remove the elastomeric film barrier, the area should be washed gently with soap and water and the film gently peeled off starting at the outer edges.

The clean dry, non-infected wound coated with the polyethylene carbonate composition is allowed to dry. The drying process takes less than one minute due to the low boiling point of the solvent, i.e. 39.7° C. The skin temperature is about 33° C., body temperature about 37° C., promoting evaporation of the solvent, and flow of the polyalkylene carbonate polymer, which has a glass transition temperature of about 20-25° C., thereby resulting in the flow of polyalkylene carbonate into the pores around the clean dry, non-infected wound.

Since a cotton tip brush-type applicator is being used for application, a polyethylene carbonate composition with an intermediate polyethylene carbonate polymer concentration is used with proper viscosity to prevent the polyethylene carbonate composition from running away from the clean dry, non-infected wound to be treated. Polyethylene carbonate polymer concentrations in this application are usually in the range of from about 7.5% to about 10% by weight of the solution. This would also apply with a glass eye-dropper or glass rod application.

While a brush type application is used in accordance with a preferred implementation of the present method, it is appreciated other modes of application, for example, spraying, gel or squeeze tube application, or emulsion application may be used assuming suitable containers can be identified.

As mentioned above, biofilms can form on any wound when planktonic bacteria are not quickly killed by the patient's own immune system, or by exogenous antibiotics or antiseptics. As such, it follows that the presence of planktonic bacteria would impair wound healing, and some planktonic bacteria are more virulent than others because they secrete powerful exotoxins that kill wound cells or alter inflammatory cells. It further follows that it is best to intervene at an early stage, for example, colonization stage so as to prevent planktonic bacteria from attaching and converting into a protective biofilm phenotype. This is much more effective than trying to kill or remove a mature biofilm in a wound.

It has been found that the present polyethylene carbonate composition, when used in the manner described above, is effective in killing the following bacterial, fungal, and viral planktonic bacteria: Escherichia coli, Pseudomonas aeruginosa, Staphylococcus aureous, MRSA (Methicillin-resistant Staphylococcus aureus), VRE (vancomycin-resistant Enterococcus), Acinetobacter baumannii, Bacillus subtilis, Clostridium sporogenes, Streptococcus pyogenes, Aspergillus niger, T. rubrum, Candida albicans, D Aspergillus brasiliensis, Herpes Simplex 1, and Herpes Zoster.

Polyethylene carbonate film barriers will not adhere to any open sores or wounds. Further, polyethylene carbonate film barriers will inherently pull away from healthy skin. As such, the polyethylene carbonate film barrier very specifically adheres to intact skin around clean dry wounds, but not to open wound areas where new epithelialization is taking place. This distinctive adherence provides protection for the wound without concern for damage to newly forming tissue.

In addition to providing a mechanism for assisting in the healing of clean dry, non-infected wounds, the polyethylene carbonate composition of the present invention provides a biodegradable dressing for the protection of clean dry, non-infected wounds as a replacement for currently existing non-degradable dressings.

Further still, the polyethylene carbonate composition of the present invention provides a biodegradable wound dressing that may be used in conjunction with various imaging procedures (for example, MRI) that might otherwise be detrimentally affected by metals within other wound dressings. In particular, currently existing wound dressings often include metals and, therefore, must be removed prior to the imaging procedure. The protection of wounds in accordance with the present invention offers a wound management system that will not adversely affect imaging procedures that might be necessary in the treatment of the patient.

With the foregoing in mind, it is readily apparent the polyethylene carbonate composition, when used in conjunction with the present methodology provides for ideal qualities of a wound dressing. In particular, the polyethylene carbonate composition when used in conjunction with the present methodology offers the following benefits:
broad antimicrobial spectrum considering the fact that biofilm is often polymicrobial, including both bacteria, fungi, and unidentified pathogens;
no microbial resistance and thereby targeting a universal trait of all biofilm microbes that cannot be genetically modified;
low microbial tolerance having a proven ability to thwart the strategies microbes use to tolerate antimicrobials;
high tissue compatibility wherein the microbicidal effects thereof do not damage healthy tissue;
elastomeric film barrier inhibiting microorganism migration into the wound thereby reducing likelihood of biofilm formation; and
well tolerated by host wound cells.

Ultimately, the present invention offers a unique and novel mechanism for preventing biofilm. The microbicidal agent (that is, methylene chloride) in the polyethylene carbonate composition is capable of killing quiescent biofilm-associated bacteria by eradicating any microbe it comes in contact with. There is no known tolerance to the polyethylene carbonate composition, meaning there is no genetic change but the bacteria are less susceptible to killing. Both resistance and tolerance may cause antimicrobials, both topical and systemic, to be ineffective against biofilm, while the polyethylene carbonate composition of the present invention has neither of the drawbacks.

The polyethylene carbonate composition of the present invention is composed of a positively-charged polymer that has been extensively studied for more than 10 years. Its mechanism of action has been well characterized. The polyethylene carbonate composition of the present invention kills microbes through direct physical contact rather than chemical reactions (as is the case for silver and most antibiotics).

The positively-charged polymer of the polyethylene carbonate composition interacts with negatively-charged phospholipids in microbial membranes, forming “punched holes” within the membrane, and thereby licing and/or eradicating the membrane. This results in loss of cellular integrity and is quickly followed by bacterial cell death, while maintaining low cytotoxicity to surrounding mammalian/host cells. Since it does not rely on cellular activity (unlike antibiotics), the polyethylene carbonate composition of the present invention is effective against quiescent cells. Further, the mechanism of action offered by the polyethylene carbonate composition of the present invention eradicates any microbial organism it comes in contact, and there is no known microbial resistance. Given that virtually all microbes have negatively-charged phospholipids in their cell membranes, the polyethylene carbonate composition of the present invention has a broad antimicrobial spectrum that includes gram-positive bacteria (including MRSA), gram-negative bacteria (including P. aeruginosa and E. coli), anaerobic bacteria, spore-forming bacteria, intracellular bacteria (including Chlamydiae and Mycoplasma), and fungi (including C. albicans and Aspergillus niger).

The polyethylene carbonate composition kills both free-floating and biofilm-associated microbes. The polyethylene carbonate composition can therefore be useful in preventing biofilm reformation which can begin within 24 hours after wound debridement. For patients who are receiving frequent debridements, the polyethylene carbonate composition can be used as a “bridge” between debridement visits as long as the wound is clean and dry with no signs of active infection.

As the preceding discussion shows, the polyethylene carbonate composition is a good first-line therapy to address bioburden. As such, and if the wound responds adequately, the application of the polyethylene carbonate composition should be continued. However, if the non-infected wound does not adequately shrink in size as expected, despite adequate bioburden control, clinicians should consider advancing therapy to bioengineered living cell-based products.

Further still, the polyethylene carbonate composition should be used to prevent biofilm reformation between debridement visits. The polyethylene carbonate composition controls bioburden, as well as supports and promotes healing.

While the method disclosed above is directed at eliminating bioburden in the prevention of biofilm in wound care, it is appreciated that biofilms are found not only in wounds, but also on medical devices such as prosthetic heart valves, orthopedic implants, intravascular catheters, and artificial hearts, to name a few. As such, it is contemplated the methods of the present invention could be applied in various areas for the purpose of killing bioburden in the prevention of biofilm.

While the preferred embodiments have been shown and described, it will be understood that there is no intent to limit the invention by such disclosure, but rather, is intended to cover all modifications and alternate constructions falling within the spirit and scope of the invention.

1. A method for wound management in prevention of bioburden and/or biofilm, comprising:
conducting an initial risk assessment protocol with regard to a wound;
washing the wound and surrounding skin;
applying a polyethylene carbonate composition on and around the wound;
eradicating any organisms as methylene chloride of the polyethylene carbonate composition evaporates and polyethylene carbonate polymer of the polyethylene carbonate composition polymerizes;
allowing time for formation of a clear elastomeric film barrier over the wound and around the wound.

2. The method according to claim 1, further including the step of releasing CO₂ into the wound as the polyethylene carbonate polymer of the polyethylene carbonate composition polymerizes and forms the elastomeric film barrier.

3. The method according to claim 1, further including the step of providing a barrier to the flow of O₂ into and from the wound after the elastomeric film barrier is formed.

4. The method according to claim 1, further including the step of visually inspecting the wound, on a daily basis, through the elastomeric film barrier to identify potential indicators of bioburden, biofilm, and/or maceration.

5. The method according to claim 1, further including the step of inspecting the elastomeric film barrier for leakage and loosening of edges.

6. The method according to claim 1, wherein the step of conducting an initial risk assessment includes visually inspecting to identify existing bioburden and prior development of biofilm, determining non-healing status of the wound, identifying personal characteristics leading to susceptibility to the development of bioburden and/or biofilm, and identifying potential medical procedures conflicting with conventional wound treatment protocols.

7. The method according to claim 1, wherein the step of washing further includes cleaning and drying the wound and surrounding skin.

8. The method according to claim 1, wherein the step of applying includes covering an area that extends at least one and one-quarter inches beyond edges of the wound with the polyethylene carbonate composition.

9. The method according to claim 1, wherein the polyethylene carbonate composition includes polyethylene carbonate polymer dissolved in methylene chloride.
